(19) United States
(12) Patent Application Publication
(10) Pub. No.: US 2013/0053719 A1
(43) Pub. Date: Feb. 28, 2013

(54) ASTHMA MONITORING DEVICE

(71) Applicant: William Oren Wekell, Maple Valley, WA (US)

(72) Inventor: William Oren Wekell, Maple Valley, WA (US)

(21) Appl. No.: 13/215,088

(22) Filed: Aug. 22, 2011

(51) Int. Cl. A61B 5/087; G06Q 50/00; A61B 5/09

(52) U.S. Cl. 600/539; 600/538; 705/3

(57) ABSTRACT

A monitoring device to be used in conjunction with a patient's airway. Some embodiments of the monitoring device include at least one sensor assembly configured to detect inhaled and exhaled airflow and an optional communications subassembly configured to transmit measurements to at least one external computing device. Other embodiments include at least one sensor assembly configured to detect a measurement indicating airflow from the patient's lungs and at least one processor configured to detect whether the airflow is associated with medication administration or measurement of breathing characteristics. Some embodiments include a circuit that allows patient information to be analyzed by an external computing device and/or the monitoring device to determine whether the patient's breathing has declined indicating a medical problem. Optionally, the monitoring device may include a display for displaying whether the airflow measurement indicates a good outcome, a potential concern, or a serious condition

### BACKGROUND OF THE INVENTION

1. Field of the Invention

The present invention is directed generally to devices and methods of monitoring patient health, and more particularly, to methods and systems of managing asthma and other airway disorders.

2. Description of the Related Art

Monitoring patient parameters is quite common in medical care environments, such as hospitals, doctors' offices, and the like. Further, patient monitoring outside of a clinical setting is increasing because of the rising cost of traditional healthcare. There is a need for devices configured to monitor a patient's health. Devices configured to inform professional healthcare providers when appropriate are particularly desirable.

Asthma is a disease characterized by airway constriction often caused by a genetic predisposition and sensitivity to environmental factors. These environmental factors or triggers, as they are sometimes called, are often idiosyncratic to a particular patient. Environmental triggers of asthma can cause chronic or acute symptoms that can range from annoying to life threatening. These symptoms typically range from coughing, wheezing, and shortness of breath to drastically decreased air exchange as measured by a spirometer or peak expiratory flow meter.

Both indoor and outdoor environments can include substances that trigger asthma. Some common indoor environmental triggers are biologic allergens, tobacco smoke, irritant chemical and fumes as well as products of combustion. Outdoor environmental triggers that are most common include O₂, SO₂, NO₂, lead, and particulate matter (e.g., particles smaller than about 10 microns).

It is important for patients to learn about their particular environmental triggers and to adapt to avoid them or minimize their effects. It is also important to determine a treatment plan with a healthcare provider to diminish or avoid the symptoms of asthma. This treatment plan needs to be personalized and maintained. The treatment plan may need to be adjusted over time because of changes in environmental conditions, a patient's response(s) to stress, and other health related issues.

During an acute asthma attack, narrowing of the patient's airway can cause severe discomfort or worse. These events are dangerous and are to be avoided if possible. But it can be difficult to manage this condition successfully. Asthma attacks can leave family and friends in the position of providing first responder care to the patient whether they know anything about the disease or not.

There are several types of medications that are used to alleviate asthma symptoms. They are generally put in two classes. The first class of medications includes drugs administered on a relatively regular basis that have a long lasting effect. The second class of medications includes drugs that are used in an acute condition and are often referred to as rescue inhalers.

Both types of medications are typically administered to the patient orally by means of a metered dose inhaler applicator and breathed into the patient's airway directly. These inhaler applicators provide measured dosages for a recommended number of applications or doses. Some sophisticated inhaler applicators provide a mechanical counter that counts the number of doses administered by the inhaler to indicate when the recommended number of doses have been administered by the inhaler applicator. The patient is able to view the counter and use it to determine whether the specified number of doses of a drug have been administered by the inhaler applicator indicating it is time to obtain more of the drug.

It is recognized that precursor symptoms often precede asthma attacks. One such precursor symptom that is widely accepted as a useful measure of health is peak expiratory airflow. Peak expiratory airflow can be used to identify problems before they become apparent to the patient. With careful monitoring of peak expiratory airflow, it may be possible to help the patient recognize impending problems and avert an emergency or lessen its severity.

If a patient's airways are constricted, it takes longer for the patient to empty the patient's lungs. The amount of time required for the patient to empty his/her lungs may be determined by having the patient blow through a device that measures maximum exhalation rate. Both mechanical and electronic devices currently exist in the marketplace that measure maximum exhalation rate. Healthcare practitioners commonly recommend the use of such devices to patients for the purposes of understanding and managing their condition.

Unfortunately, it is inconvenient to carry and/or use such devices for airflow measurement. It is also not easy to track and trend medication usage for review by the healthcare provider. Further, it is difficult to determine how many applications of a medication have been dispensed by an inhaler applicator. And, it is difficult to know whether a child or elderly person is administering his/her medication according to a treatment plan. Therefore, a need exists for methods and systems that track a patient's physiological parameters (e.g., peak expiratory airflow) and medication administration to help manage asthma in chronic and/or acute conditions.

The present application provides these and other advantages as will be apparent from the following detailed description and accompanying figures.

BRIEF DESCRIPTION OF THE SEVERAL VIEWS OF THE DRAWING(S)

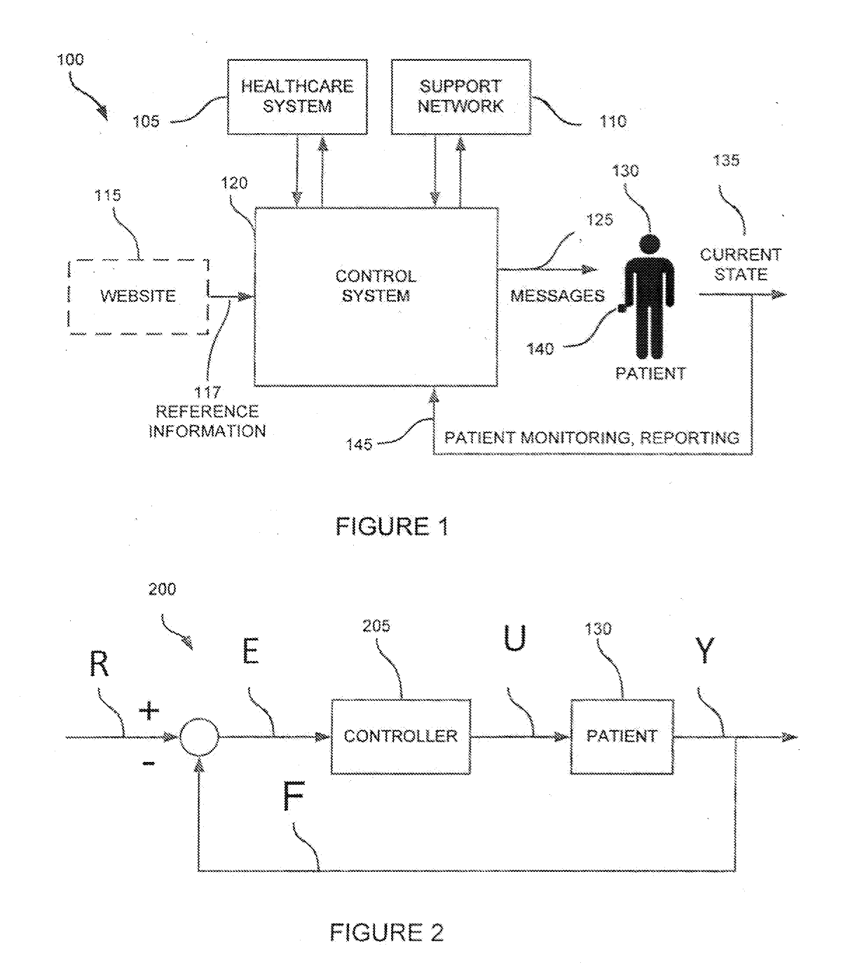

FIG. 1 is a diagram of a system configured to manage asthma.

FIG. 2 is a diagram of a control loop implemented by the system of FIG. 1.

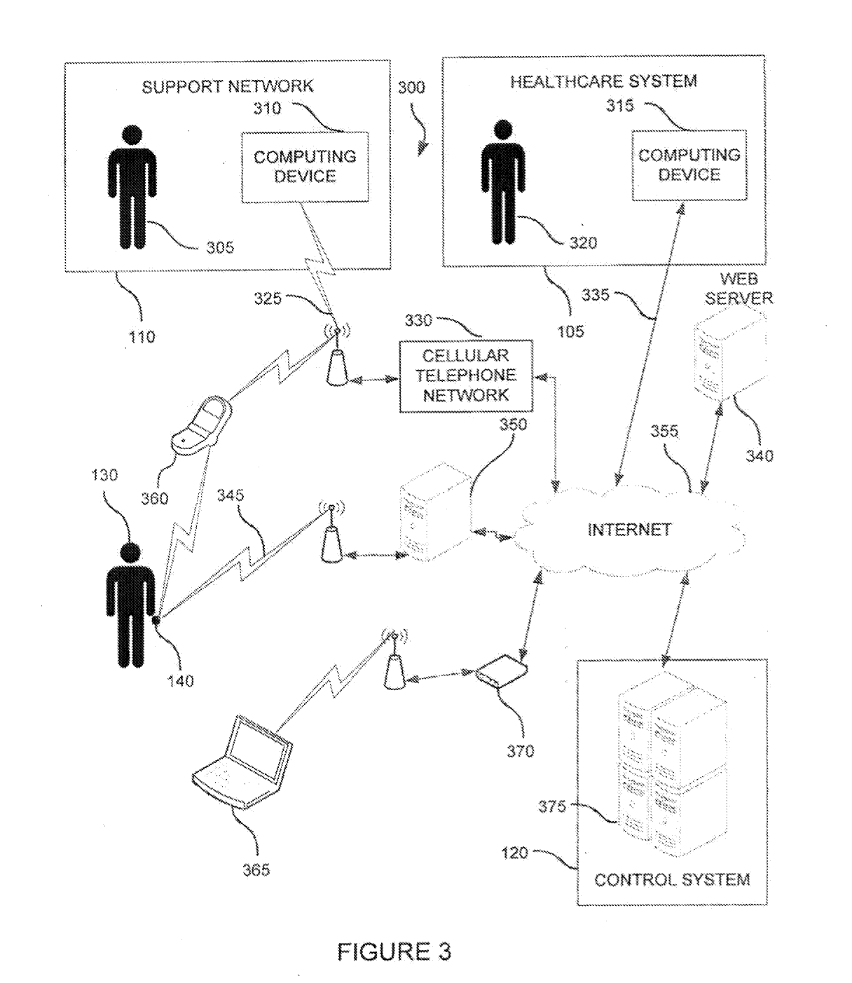

FIG. 3 is a diagram of an embodiment of the system of FIG. 1.

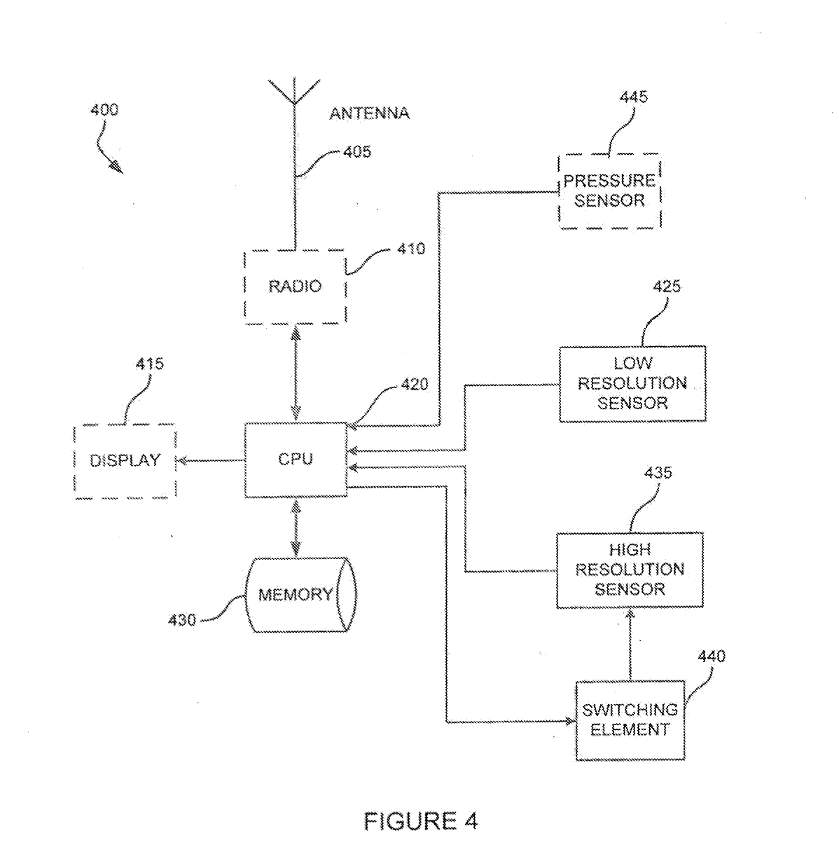

FIG. 4 is a diagram of an exemplary circuit that may be used to construct a medication dispensing device to collect inhaler medication data and airflow data.

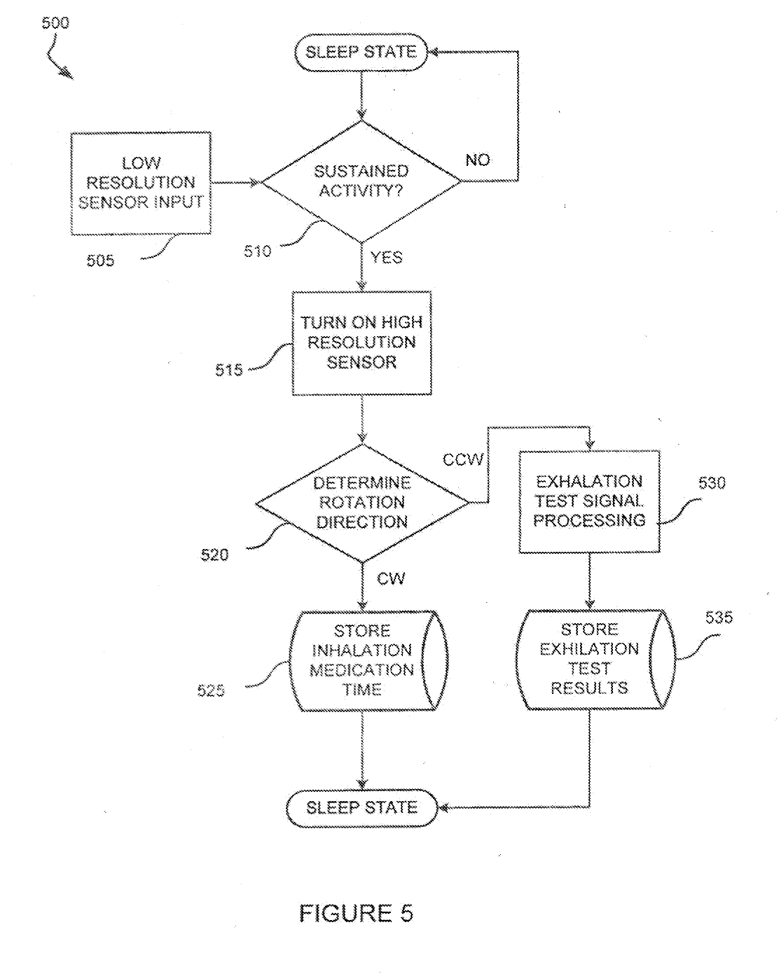

FIG. 5 is a flow diagram of a method for determining direction of airflow.

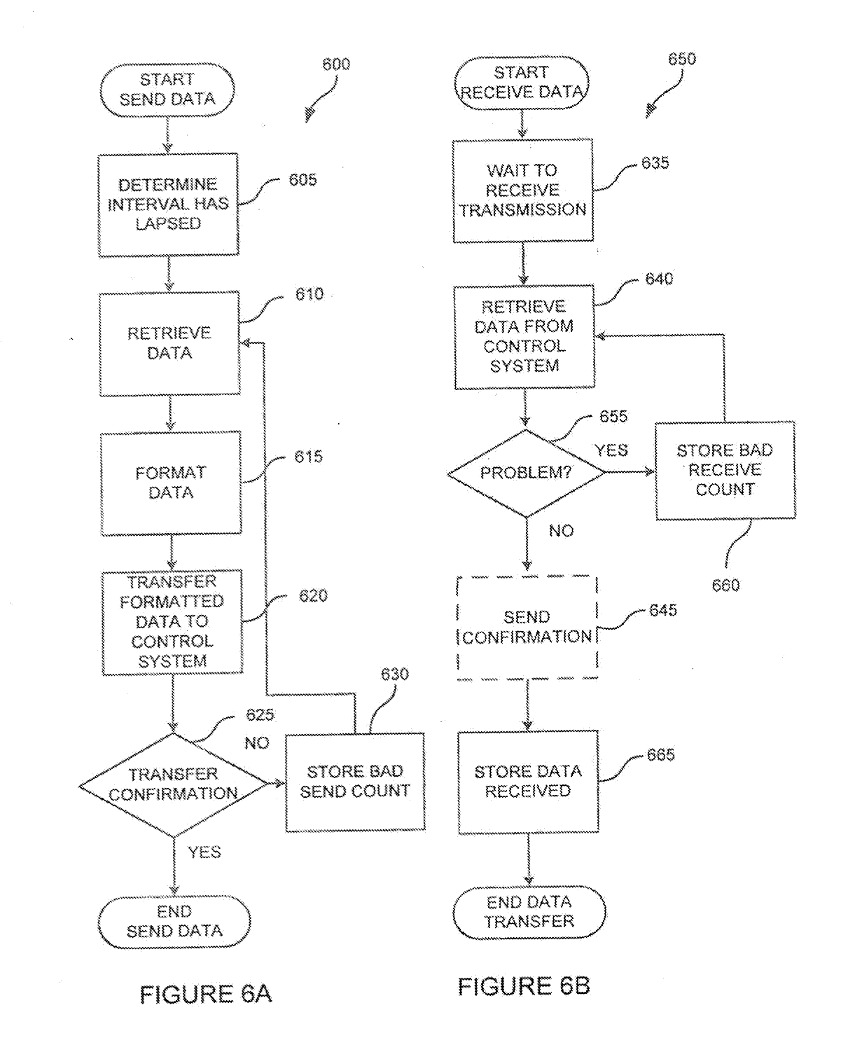

FIG. 6A is a flow diagram of a method of transmitting patient data collected to the control system.

FIG. 6B is a flow diagram of a method performed by the medication dispensing device when receiving messages and or data from the control system.

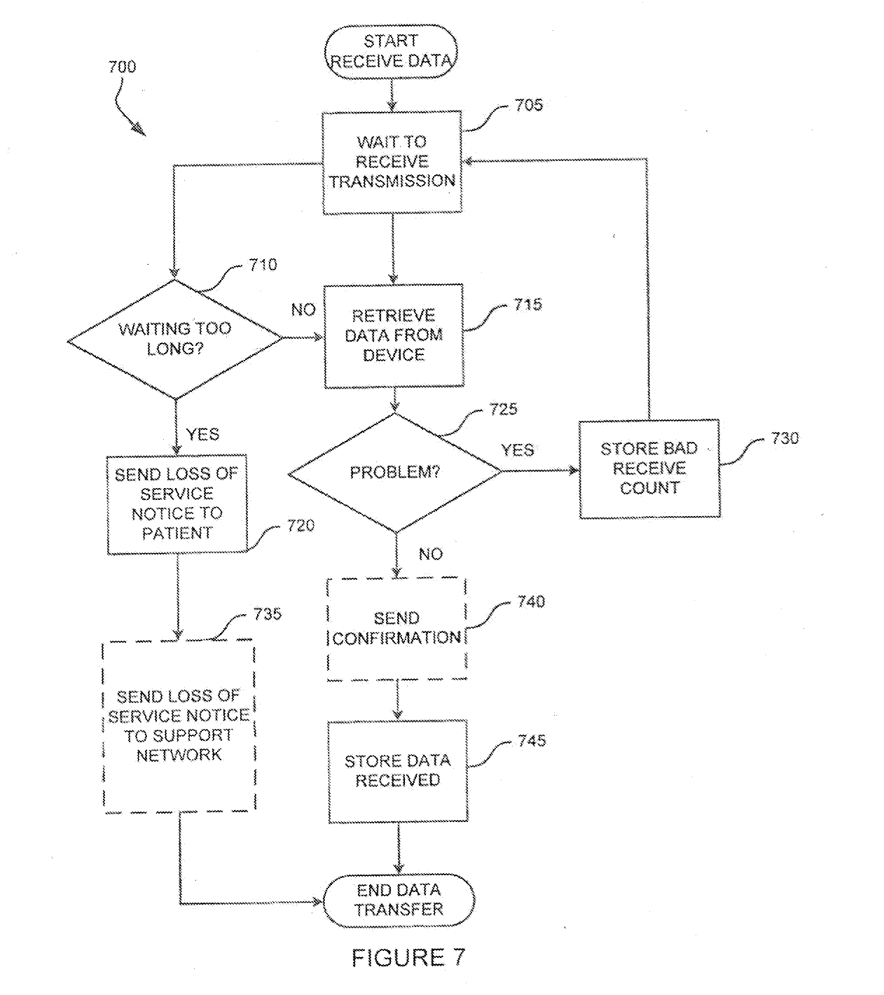

FIG. 7 is a flow diagram of a method performed by the control system when receiving the patient data from the medication dispensing device.

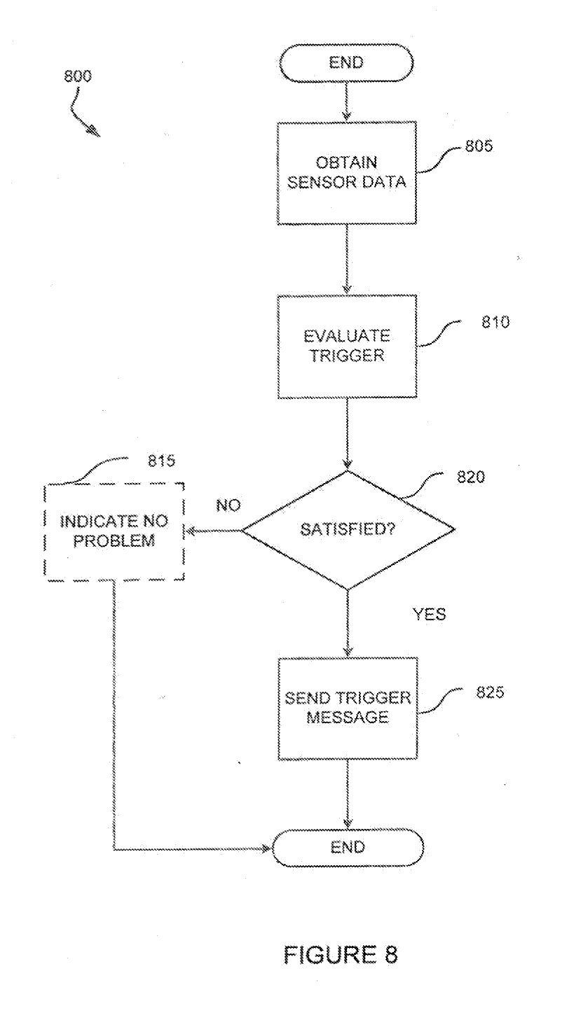

FIG. 8 is a flow diagram of a method of analyzing the patient data performed by the control system.

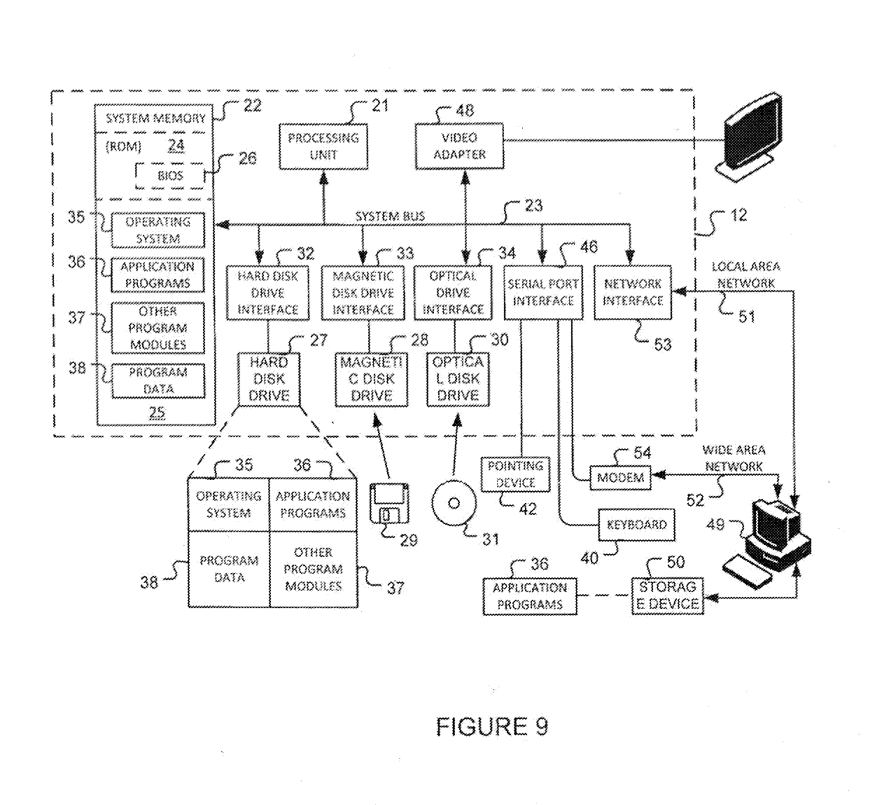

FIG. 9 is a diagram of a hardware environment and an operating environment in which one or more of the computing devices of the system of FIG. 1 may be implemented.

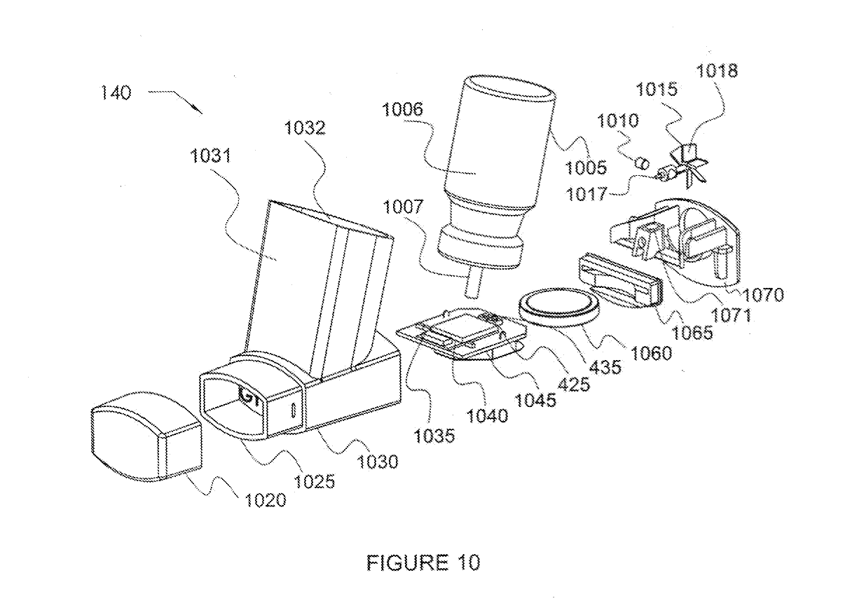

FIG. 10 is an exploded perspective view of an embodiment of the medication dispensing device.

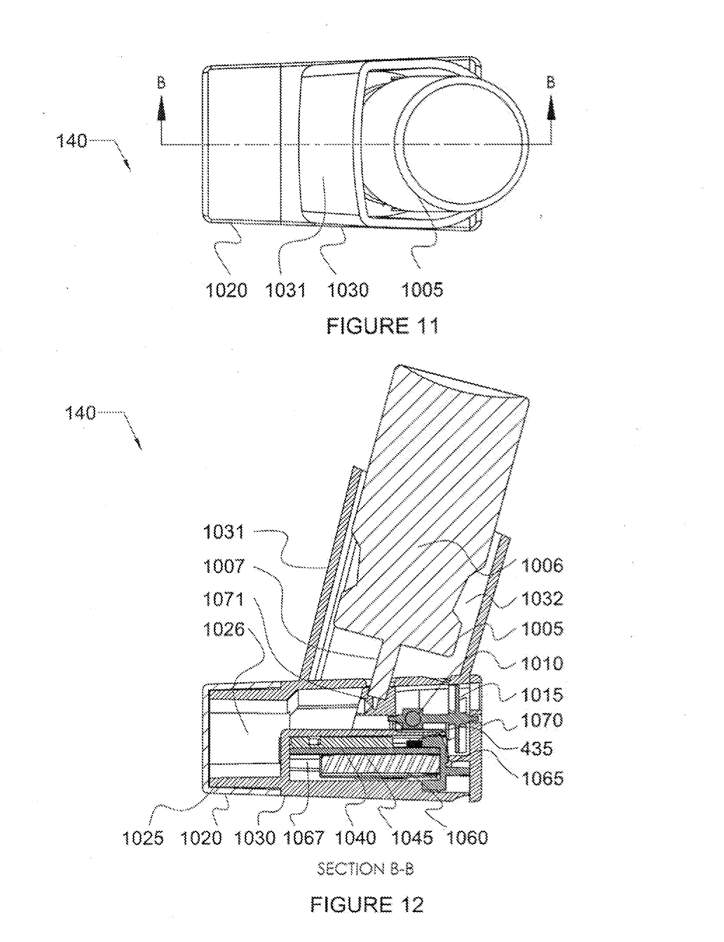

FIG. 11 is a top view of the medication dispensing device shown in FIG. 10.

FIG. 12 is a cross-sectional view of the medication dispensing device taken along sectioned line B-B of FIG. 11.

### DETAILED DESCRIPTION OF THE INVENTION

### Overview

The present invention is directed generally to devices and methods of monitoring patient health, and more particularly, to methods and systems of managing asthma and other airway disorders including, for example, chronic obstructive pulmonary disease (COPD) or other diseases or conditions relating to the respiratory system. For illustrative and explanatory purposes, the description below describes embodiments of the present invention with regard to treating asthma conditions, but it will be appreciated by a person of ordinary skill in the art that the present invention is applicable to other airway disorders as well.

Referring to FIG. 1, the present application describes a system 100 that includes a device 140 used by a patient 130 that is connected (e.g., wirelessly) to a control system 120. The control system 120 may be connected (e.g., wirelessly) to a healthcare system 105, a support network 110, and the like. The healthcare system 105 includes healthcare professionals, physicians, hospitals, pharmacies, and the like. The support network 110 includes the patient's friends, family, as well as others involved in the patient's care. The patient 130, support network 110, and/or the healthcare system 105 may provide reference information 117 to the control system 120. The reference information 117 may be used to setup or configure the control system 120. By way of a non-limiting example, the reference information 117 may include patient information (e.g., age, height, weight, etc.), patient diagnosis, past asthma attack triggers, weather, pollen count, ozone levels, air pollution, message routing information, and trigger values. The reference information 117 may also include instructions (e.g., patient instructions) associated with the trigger values. Such instructions may include a predetermined prescribed treatment plan (e.g., instructions to increase a dosage or use a particular type of inhaler medication), instructions to perform airflow tests over a given time period, requests for patient symptom information, instructions to contact a healthcare professional, and the like. The reference information 117 may have been provided to a website 115 generated by an optional web server 340 (illustrated in FIG. 3) and forwarded to the control system 120 by the web server 340.

The control system 120 may issue messages 125 to the patient 130 (e.g., via the patient's cell phone, or the like) that could cause a modification in the patient's state 135 (e.g., a reduction in asthma symptoms, a reduction in the likelihood of an asthma attack, and the like). When triggered by trigger values, the messages 125 may include one or more instructions associated with the trigger values.

The device 140 is configured to be used by the patient 130 for the administration of inhaler medications and for the measurement of airflow. When used in this manner, the device 140 occasionally collects data that may be processed by the control system 120 to obtain medication administration information and peak expiratory airflow information. The data collected by the device 140 is sent to the control system 120 in a device message 145. In particular implementations, the device 140 is configured to determine both an amount of medication administered and the patient's peak expiratory airflow and to send this information to the control system 120 in a device message 145. In other implementations, the device 140 is configured to determine that medication has been administered by the device 140 and the patient's peak expiratory airflow and to send this information to the control system 120 in a device message 145. The control system 120 analyzes the data received from the device 140 in the device message 145 to determine the medication administration information and peak expiratory airflow information.

Over time, multiple airflow measurements may be collected and tracked for the purposes of detecting a trend in medication administration, airflow, or patient airway restriction. The previously obtained airflow measurements and medication administration events may be stored in the reference information 117.

The healthcare provider or system 105 may access the control system 120 to review the airflow measurement(s) to detect potential problems and/or recommend treatments or changes in treatment. Further, when the control system 120 detects a trend or sudden change of the airflow capacity, the control system 120 may send messages to the healthcare system 105, the support network 110, the patient 130, and the like. Preconfigured system trigger values may be used to detect a trend or sudden change of the airflow capacity. Further, one or more instructions may be associated with each of the system trigger values. When triggered by a preconfigured system trigger value, messages sent to the healthcare system 105, the support network 110, the patient 130, and the like may include the one or more instructions associated with the system trigger value.

By way of a non-limiting example, a juvenile might take a peak expiratory flow measurement that is lower than a preconfigured trigger value, such as 60% of the maximum recorded peak expiratory flow measurement. This measurement would be transmitted a server of the system for analysis and would be compared to patient history and preconfigured trigger values. A low measurement compared to a trigger value might launch several preconfigured actions. The first might be to send a message to the juvenile's phone (e.g., the patient 130) instructing the juvenile to administer a medication, limit activity, or limit exposure to an irritant. A second preconfigured message might be to the parent's cellular phone or computing device (e.g., the support network 110) informing them of a problem that has occurred that requires their attention. Additionally, a preconfigured message for the healthcare professional (e.g., the healthcare system 105) might be sent such that when the doctor reviews the asthma treatment plan and patient history, dear deviations are readily apparent.

Likewise, the absence of event tracking information might inform a user or caregiver of a missed scheduled medication administration or measurement event, or even a prolonged loss of contact that may indicate a device failure. In some embodiments, this would trigger a message to the appropriate individual(s) in charge of a patient's care.

Messages sent to the healthcare system 105, the support network 110, and/or the patient 130 may include SMS cellular telephone messages, recorded voice messages (e.g., including educational information), alerts, alarms, and the like.

Thus, the system 100 provides a means of assessing changes in the medicine administration and the peak expiratory airflow of a patient and more particularly the onset of asthma symptoms and attacks. The assessment may be conducted remotely by the control system 120 and/or the healthcare system 105. Members of a support network 110 such as parents and/or the healthcare system 105 need not be present to collect or evaluate the airflow measurement. Instead, airflow measurements may be collected automatically by the device 140 and optionally, transferred (e.g., wirelessly) to the system 100. Medicine administration and airflow measurements may be collected on an ongoing basis over any desired length of time.

The system 100 may be conceptualized as a continually readjusting system that seeks a stable desired condition (e.g., an absence of asthma symptoms). The system 100 may implement a control loop 200 illustrated in FIG. 2. In the control loop 200, reference information “R” is provided and compared with feedback information “F.” With respect to the system 100, the reference information “R” may be the reference information 117 provided to the control system 120, and the feedback information “F” may be the device messages 145 transmitted by the device 140.

The difference between the reference information “R” and the feedback information “F” is an error “E.” The error “E” is input into a controller 205.

In system 100, the error “E” is calculated by the control system 120. The controller 205 in turn issues commands “U” (e.g., the messages 125) that are used to affect the state of the patient 130. In the system 100, the control system 120 may issue the messages 125 to the patient 130, the support network 110, and/or the healthcare system 105. This is reflected in a current state “Y.” In FIG. 1, the patient's current state is labeled with reference numeral 135. The current state “Y” provides the feedback information “F” that is compared to the reference information “R.” In other words, the patient's state 135 provides the device messages 145 (with data used to obtain the airflow measurement) that are compared to the reference information 117 (e.g., previously obtained airflow measurements). Based on the results of this comparison, one or more messages 125 may be sent to the patient 130, the support network 110, and/or the healthcare system 105 to modify the patient's state 135 by modifying his or her behavior.

### System 300

FIG. 3 illustrates a system 300, which is an exemplary implementation of the system 100 (see FIG. 1). Turning to FIG. 3, the control system 120 includes a database server 375. The reference information 117 (see FIG. 1) is stored in the database server 375. The reference information 117 may be received by the database server 375 during an initial setup process as well as on an ongoing basis. The system 300 may include the optional web server 340 configured to generate the website 115 (see FIG. 1) to which the reference information 117 may be provided and transferred to the database server 375 (e.g., over the Internet 355 or other network) of the control system 120. The database server 375 may also store pertinent data about the patient 130 (such as patient history, patient record, and the like), trigger event levels (discussed below), and addresses to which messages (e.g., notifications, alerts, and the like) are to be sent.

Feedback information (e.g., the device message 145 illustrated in FIG. 1) most often originates from the patient 130 and/or the device 140. This feedback information can travel several alternate paths depending upon the implementation details. For example, the feedback information may be input into a computing device (e.g., a patient desktop computer 350, a patient cellular telephone 360, a patient portable computer 365, and the like) connected to the database server 375 (or the web server 340) via the Internet 355. The device 140 may communicate with the computing devices via a wired or wireless communication link (e.g., a communication link 345). Over a wireless communication link, the device 140 may communicate with the computing devices using SMS messages, WIFI protocols, Bluetooth protocols, and the like. The computing devices may transfer the feedback information to the database server 375 of the control system 120. The device 140 may communicate the device messages 145 to the computing devices for subsequent transmission thereby to the database server 375.

In the embodiment illustrated, the patient desktop computer 350 is connected to the Internet 355 via a conventional wired connection. In the embodiment illustrated, the patient cellular telephone 360 and the patient portable computer 365 are connected to the Internet 355 by an Internet gateway device 370 (e.g., a modem). The patient cellular telephone 360 and the patient portable computer 365 may also communicate with the Internet gateway device 370 using WIFI protocols, Bluetooth protocols, and the like.

By way of a non-limiting example, the feedback information may be transmitted by the device 140 via a radio link (e.g., the radio link 345) to the patient desktop computer 350, the patient cellular telephone 360, the patient portable computer 365, and the like. By way of another non-limiting example, the feedback information may be transmitted by the device 140 directly to the Internet gateway device 370.

The feedback information is received by the database server 375. In the embodiment illustrated, the database server 375 implements the control system 120 (see FIG. 1) that compares the current state 135 of the patient 130 and the reference information 117 (which may include previously received feedback information).

One or more messages 125 (see FIG. 1) to be reviewed by the patient 130 may be transmitted by the database server 375 to the device 140, the patient desktop computer 350, the patient cellular telephone 360, the patient portable computer 365, and the like. By way of a non-limiting example, such messages may be displayed on the website 115 (see FIG. 1) generated by the web server 340. In such embodiments, the database server 375 is configured to instruct the web server 340 to display messages on the website 115. The patient desktop computer 350, the patient cellular telephone 360, and/or the patient portable computer 365 may connect to the web server 340 over the Internet 355 and display the website 115 using a conventional web browser application.

The support network 110 may include one or more computing devices (e.g., a support network computing device 310) connected to the database server 375 via the Internet 355. One or more messages to be reviewed by a support person 305 (e.g., a parent of a patient) may be transmitted by the database server 375 to the computing device 310. By way of a non-limiting example, such messages may be displayed on the website 115 (see FIG. 1) generated by the web server 340. In the embodiment illustrated, the computing device 310 is connected to the Internet 355 via a wireless communication link 325 with a cellular telephone network 330. The computing device 310 may connect to the web server 340 over the Internet 355 and may display the website 115 using a conventional web browser application. Some patients may rely on help from the support network 110 while others may have no such support.

The healthcare system 105 may include one or more computing devices (e.g., a healthcare provider or caregiver computing device 315) connected to the database server 375 via the Internet 355. One or more messages to be reviewed by a caregiver 320 may be transmitted by the database server 375 to the computing device 315. By way of a non-limiting example, such messages may be displayed on the website 115 (see FIG. 1) generated by the web server 340. In the embodiment illustrated, the computing device 315 is connected to the Internet 355 via a wired communication link 335. The computing device 315 may connect to the web server 340 over the Internet 355 and may display the website 115 using a conventional web browser application.

A diagram of hardware and an operating environment in conjunction with which implementations of the database server 375, the patient desktop computer 350, the patient cellular telephone 360, the patient portable computer 365, the support computing device 310, the caregiver computing device 315, and the web server 340 may be practiced is provided in FIG. 9 and described below.

### Circuit 400

FIG. 4 is a block diagram illustrating example electrical components of the device 140. The electrical components of the device 140 include a circuit 400, which includes a low resolution sensor 425, a high resolution sensor 435, a switching element 440, a memory 430, an antenna 405, a radio 410, a display 415, a processor 420 (e.g., a CPU), and an optional pressure sensor 445.

The low and high resolution sensors 425 and 435 are each operative to detect magnetic fields. In the embodiment illustrated, the low and high resolution sensors 425 and 435 use a Hall Effect to sense a magnetic field and are configured to output a digital signal in the presence of a perpendicular magnetic field of suitable intensity. Further, the low and high resolution sensors 425 and 435 include digital circuitry that cycles power and retains state information. The low resolution sensor 425 may cycle less frequently to reduce power consumption at the expense of resolution.

In some embodiments, the low resolution sensor 425 remains powered continuously and the processor 420 is typically in a sleep state, continuously powered to retain data stored in the memory 430. Magnetic activity of suitable strength will initiate a signal from the low resolution sensor 425 to the processor 420. The processor 420 will then communicate to the switching element 440, which will provide power to the high resolution sensor 435. The operation of the circuit 400 is discussed below.

### Methods

As will be explained in detail below, whether the patient is inhaling or exhaling into the device 140 may be determined based on a direction of rotation of a magnetic element (e.g., a magnet 1010 illustrated in FIG. 10) coupled to a rotatable body (e.g., a rotatable body 1015 illustrated in FIG. 10). The rotatable body is configured to rotate the magnetic element in both a clockwise “CW” direction and a counter clockwise direction “CCW.” The low and high resolution sensors 425 and 435 shown in FIG. 4 are configured to sense the magnetic field of the magnetic element.

FIG. 5 is a flow diagram of a method 500 of determining whether the patient is inhaling into the device 140 (taking medication) or exhaling into the device 140 (measuring airflow). Before the method 500 begins, the processor 420 is in a sleep state. In a first block 505, the state of the low resolution sensor 425 is changed by the rotation of the magnetic element. The state of the low resolution sensor 425 changes when the sensor detects a perpendicular magnetic field of suitable intensity. Thus, when the magnetic element (e.g., a magnet 1010 illustrated in FIG. 10) is being rotated by the rotatable body (e.g., a rotatable body 1015 illustrated in FIG. 10) relative to the low resolution sensor 425, the sensor changes state. Changes in state of the low resolution sensor 425 are transmitted to the processor 420. The processor 420 evaluates the state change in decision block 510 to determine whether the change is sustained activity or hysteresis. A change in the magnetic field sense state of the low resolution sensor 425 changes a voltage level on an interrupt line connected to the processor 420. This voltage change brings the processor 420 out of a low power state. The processor 420 then looks for additional changes in the voltage of the low resolution sensor 425. If no additional state changes occur in a predetermined period, the processor 420 returns to a low power state.

If activity is not sustained, the decision in the decision block 510 is “NO,” and the processor 420 returns to a sleep state. On the other hand, if activity is sustained, the decision in the decision block 510 is “YES,” and in block 515, the switching element 440 activates the high resolution sensor 435.

In decision block 520, the processor 420 determines whether the magnet is rotating clockwise “CW” or counter clockwise “CCW.” By knowing the physical geometry of the device 140 and comparing the timing of signals from the low and high resolution sensors 425 and 435, the processor 420 may determine signal timing precedence from the sensors 425 and 435 and infer the direction of rotation of the magnetic element.

If in decision block 520, the processor 420 determines the magnetic element is rotating clockwise “CW,” the processor 420 advances to block 525. In the embodiment illustrated, air moving through the device 140 toward the patient 130 causes the magnetic element to rotate in a clockwise “CW” direction. In such embodiments, when the magnetic element is rotating in a clockwise “CW,” the patient 130 is inhaling. This inhalation is assumed to be a medication dispensing event and in block 525, the time the inhalation occurred is stored in the memory 430 (see FIG. 4). The processor 420 then returns to a sleep or wait state.

If, in decision block 520 the processor 420 determines the magnetic element is rotating counter clockwise “CCW,” the processor 420 advances to block 530. In the embodiment illustrated, air is moving though the device away from the patient 130 causes the magnetic element to rotate in a counter clockwise “CCW” direction. In such embodiments, when the magnetic element is rotating in a counter clockwise “CCW,” the patient is exhaling (i.e., testing expiratory flow rate). Timing measurements are taken of the rotation event to determine characteristics of the exhalation, such as airflow rate.

By capturing the speed and direction of the rotating body 1015 many times a second as it rotates, the rate of airflow at different times during the exhalation and subsequent inhalation events may be measured, stored, integrated, analyzed, or transmitted. A comparison of these airflow measurements over time can be useful to a healthcare provider in understanding the disease state. Tests can be initiated that measure both inhalation as well as exhalation characteristics when the events are continuous. Many possible spirometry measurements are possible. Some of these measurements might include forced expiratory flow, forced expiratory volume in a time period (e.g., 1 second), forced expiratory volume ratios, forced vital capacity, tidal volume, and the like.

In a non-limiting example, peak expiratory flow rate is calculated in block 530. In block 535, the results of the direct measurements or calculations are stored in the memory (see FIG. 4). Then, the processor 420 turns off the high resolution sensor 435 and returns to a sleep state. Optionally, the processor 420 may transmit the data via the radio 410 and the antenna 402 to the control system 120. Then, the method 500 terminates.

FIG. 6A is a flow diagram of a method 600 of transferring data from the device 140 to the control system 120. When the method 600 begins, the processor 420 is in the wait state. In block 605, the processor 420 determines a predetermined transmission interval has lapsed since a transmission was last sent by the device 140 to an external device (e.g., the patient desktop computer 350, the patient cellular telephone 360, the patient portable computer 365, the Internet gateway device 370, and the like). As discussed above, the external device is configured to transfer the data sent to it by the device 140 to the control system 120. In other embodiments, the device 140 may send data to the control system 120 without using an intermediate device. In block 610, the processor 420 retrieves the rotational data, airflow rate data, time encoding data, medication type, dosages, expiration dates, and/or other data stored in the memory 430 (see FIG. 4) during blocks 525 and 535 of method 500 (see FIG. 5). In block 615, the information retrieved is formatted for transmission (e.g., placed in transmission packets) and subsequently transferred to the external device or control system 120 in block 620.

In decision block 625, the processor 420 determines whether a transfer confirmation has been received from the external device. The decision in decision block 625 is “YES” when the device 140 has received a transfer confirmation from the external device in response to the transmission sent in block 620. Otherwise, the decision in decision block 625 is “NO” when the device has not received a transfer confirmation from the external device in response to the transmission sent in block 630. Optionally, even if a transfer confirmation is received, the decision in decision block 625 may nevertheless be “NO,” if the processor 420 determines that a problem occurred when the calculated values were transmitted in block 620. When the decision in decision block 625 is “YES,” the method 600 terminates. When the decision in decision block 625 is “NO,” in block 630, the processor 420 stores bad send data in the memory 430 for analysis. Then, after a delay, the processor 420 returns to block 610.

FIG. 6B is a flow diagram of a method 650 performed by the processor 420. The method 650 is performed when the device 140 receives data from the control system 120 (e.g., through an external device). The device 140 may receive data from the control system 120 such as dosage count information following the successful transfer of data (e.g., the airflow values) to the control system 120. In first block 635, the processor 420 waits to receive a transmission from the control system 120. In block 640, the device 140 receives data, such as a setting value, or the like, from the control system 120. In block 655, the processor 420 determines whether a problem occurred when the data was received from the control system 120. For example, if too much time has elapsed since data was last received from the control system 120, the processor 420 may determine a problem occurred. The decision in decision block 655 is “YES” when the processor 420 determines a problem has occurred. On the other hand, the decision in decision block 655 is “NO” when the processor 420 determines a problem has not occurred.

When the decision in decision block 655 is “YES,” in block 660 the processor 420 stores data related to the problem (e.g., increments a bad receive count value). Then, the processor 420 returns to block 640 to wait for additional transmissions from the control system 120. By way of a non-limiting example, the control system 120 may retransmit data to the device 140 if the control system 120 determines the data was not received by the device. When the decision in decision block 655 is “NO,” in optional block 645 the processor 420 may send a receipt confirmation to the control system 120 confirming the data was received. Then, in block 665, the device 140 stores the data received and the method 650 terminates.

FIG. 7 is a flow diagram of a method 700 of receiving the calculated flow rate values at the control system 120 shown in FIG. 1. By way of a non-limiting example, the method 700 will be described as being performed by the database server 375 (see FIG. 3). In first block 705, the database server 375 waits to receive a transmission from the device 140 (e.g., via one of the external devices). In decision block 710, the database server 375 determines whether it has been waiting too long (e.g., longer than a predetermined amount of time) indicating there may be a problem with the device 140 or, in embodiments of the device 140 configured to communicate wirelessly, that the device 140 was positioned outside a radio coverage area. The decision in decision block 710 is “YES” when the database server 375 has been waiting too long for a transmission from the device 140. On the other hand, the decision in decision block 725 is “NO” when the database server 375 has not been waiting too long for a transmission from the device 140.

When the decision in decision block 710 is “YES,” the database server 375 has been waiting too long for a transmission from the device 140 and, in block 720, sends a notification indicating a problem has occurred to the device 140, the patient desktop computer 350, the patient cellular telephone 360, and/or the patient portable computer 365 to be reviewed by the patient 130. In optional block 735, the database server 375 may send a notification to the support network 110 and/or the healthcare system 105 indicating a problem has occurred.

When the decision in decision block 710 is “NO,” the database server 375 continue to wait and in block 715, receives the transmission including data or calculated values data obtained from the device 140. In decision block 725, the database server 375 determines whether a problem occurred when the data or calculated values received from the device 140. For example, the calculated value for the high resolution sensor 435 may be compared to a valid range stored in the database server 375. If the calculated value for the high resolution sensor 435 is outside the valid range, the database server 375 may determine a problem occurred. The decision in decision block 725 is “YES” when the database server 375 determines a problem has occurred. On the other hand, the decision in decision block 725 is “NO” when the database server 375 determines a problem has not occurred.

When the decision in decision block 725 is “YES,” in block 730 the database server 375 stores data related to the problem (e.g., increments a bad receive count value). Then, the database server 375 returns to block 705 to wait for additional transmissions from the device 140. By way of a non-limiting example, the device 140 may retransmit data to the control system 120 if the device 140 determines the formatted data was not received by the control system.

When the decision in decision block 725 is “NO,” in optional block 740 the database server 375 may send a receipt confirmation to the device 140 confirming the data and calculated values were received. In block 745, the database server 375 stores the data and calculated values received. Then, the method 700 terminates. The data and calculated values may be stored in a patient record associated with the device 140. By way of a non-limiting example, the transmission received by the control system 120 may include a device identifier associated with the device 140 that may be used to identify the patient record associated with the device.

FIG. 8 is a flow diagram of a method 800 of processing triggers specified for sensors other than the low and high resolution sensors 425 and 435. The method 800 may be performed by the device 140, the control system 120, and/or a combination thereof. For ease of illustration, the method 800 will be described as being performed by the database server 375 but could also be considered to be on the patient's cellular phone 360 or other device. As mentioned above, the patient 130, the support person 305 (e.g., parent) and/or the caregiver 320 may use the website 115 to specify trigger conditions (e.g., threshold values) that trigger messages to the patient 130, the support person 305, and/or the caregiver 320. The method 800 may also be used with data input by the patient 130, support person 305 or the caregiver 320. The method 800 may be used with the calculated values obtained from the sensors 425 and 435 combined with the data from one or more other sensors or data available on the patient cell phone 360 or other computing device in the control system 120. Further, data on the internet 355 such as, but not limited to, temperature, weather conditions, humidity, wind, rain, ozone level, air pollution, pollen count, air quality index, and the like may be integrated into the evaluate trigger block 810 localized by the location of a user (e.g., as indicated by his or her cellular phone location).

In the first block 805, the database server 375 obtains the relevant sensor data. In block 810, the database server 375 analyzes the sensor data relative to one or more triggers to determine whether any of the triggers have been satisfied such that a trigger message is to be sent to the patient 130 (e.g., the message 125 illustrated in FIG. 2), the support person 305, and/or the caregiver 320. For example, a trigger may have been entered into the website 115 indicating that if the patient's medication administration rises above a specified level, a trigger message is to be sent to the patient 130, the support person 305, and/or the caregiver 320 reporting unusual activity. By way of another example, increased airway obstruction might trigger a predetermined prescribed treatment plan that could include increasing a dosage of maintenance or rescue inhaler medication. By way of yet another example, sensor measurements (e.g., measurements indicating increased difficulty in breathing via peak expiratory airflow indication) may trigger additional stress testing, automated patient symptom questions, a nurse to call or messages to setup an appointment with a healthcare provider. In block 820, the database server 375 may try to identify a trend indicative of a problem.

In decision block 820, the database server 375 determines whether one or more triggers are satisfied, indicating a problem. The decision in decision block 820 is “YES” when the database server 375 determines one or more triggers are satisfied. In this case, in block 825 a trigger message is sent to the device 140, the patient desktop computer 350, the patient cellular telephone 360, the patient portable computer 365, and the like. On the other hand, the decision in decision block 820 is “NO” when the database server 375 determines none of the triggers is satisfied. When the decision in decision block 820 is “NO,” in optional block 815 the database server 375 may send a notification indicating no problem has been detected to the device 140, the patient desktop computer 350, the patient cellular telephone 360, the patient portable computer 365, and the like. The notification may be viewable on the website 115 (see FIG. 1). Optionally, the database server 375 may send a notification indicating no problem has been detected to the computing device 310 to be viewed by the support person 305 and/or to the computing device 315 to be viewed by the caregiver 320. Any such notifications may be viewable on the website 115 (see FIG. 1). Then, the method 800 terminates.

### Device 140

As described above, FIG. 4 illustrates the circuit 400 that may be used to construct the device 140. Referring now to FIGS. 10-12, various components that may be used to construct the device 140 are described.

Turning to FIG. 12, in the embodiment illustrated, the circuit 400 (see FIG. 4) is mounted on a substrate 1045 (e.g., a printed circuit board) housed inside a two-part electronics enclosure—a main enclosure 1030 and a frame 1070. A removable battery 1060 may provide power to the circuit 400, but it is understood that other power means such as photovoltaic, capacitance, energy harvesting, combination thereof, and the like may be used instead of or in addition to the removable battery 1060. In the embodiment illustrated, the processor 420, memory 430, optional radio 410, and antenna 405 are integrated in a module 1040 that is mounted on the substrate 1045. The module 1040 communicates with the optional display 415, which in this embodiment includes light emitting diodes (“LEDs”) 1035 that display green, yellow, or red depending upon the airflow measurements obtained. By way of a non-limiting example, the LEDs 1035 may include a green LED that when illuminated produces green light, a yellow LED that when illuminated produces yellow light, and a red LED that when illuminated produces red light.

The colors green, yellow, and red are commonly understood asthma descriptions of peak exhalation airflow significance. Green is interpreted as a good outcome, yellow is interpreted as a potential concern, and red is interpreted as a serious condition.

In some embodiments, the processor 420 compares a current peak expiratory airflow measurement to a previously obtained peak expiratory airflow measurement (e.g., the highest peak expiratory airflow measurement stored in the memory 430). The previous peak expiratory airflow measurement is used as a comparison measurement. If the current peak expiratory airflow measure is greater than 80% of the comparison measurement, the green LED is illuminated, indicating no problems. When the current peak expiratory airflow measure is higher than the highest peak expiratory airflow measure stored in the memory 430, the processor 420 replaces the highest peak expiratory airflow measure with the current peak expiratory airflow measure. If the current peak expiratory airflow measurement is between 50% and 80% of the comparison measurement, the yellow LED is illuminated, indicating that there may be a problem with the patient's expiratory flow rate. If the current peak expiratory airflow measurement is lower than 50% of the comparison measurement, the red LED is illuminated.

All of the LEDs may additionally be blinked to inform the patient 130 of additional conditions such as the end of a specified dosage. The red, yellow, and green LEDs may be spaced sufficiently far apart from one another to avoid miscommunication for color blind individuals. Further, different illumination patterns may be used for different LEDs. For example, the green LED may be continuously illuminated, the yellow LED may blink slowly when illuminated, and the red LED may blink faster than the yellow LED when illuminated. This will further reduce misreads and express the urgency of a red indication.

Although, this color coding is described with respect to some embodiments of the optional display 415 of the device 140, this color coding may also be provided by the display of the user cellular phone 360, the display of the desktop computer 350, the display of the portable computer 365, the display of the support network computing device 310, and/or the display of the healthcare system computing device 315. Further, the display 415 of the device 140 may include other display elements instead of or in addition to the LEDs 1035, such as a liquid crystal display (LCD), or the like.

The low resolution sensor 425 and the high resolution sensor 435 may be mounted on the substrate 1045. The low and high resolution sensors 425 and 435 are positioned to interact with the magnet 1010 that is mounted on a shaft 1017 of the rotatable body 1015. When the rotatable body 1015 is rotated, the shaft 1017 and the magnet 1010 rotate therewith as a unit. The magnet 1010 is magnetized diametrically such that north and south pole fields alternately sweep across the low and high resolution sensors 425 and 435 during rotation of the rotatable body 1015. The rotatable body 1015 has vanes 1018 to transfer energy from the airflow to move the rotatable body 1015 in a clockwise or counter clockwise rotation depending on the direction (toward or away from the patient 130) of the airflow and speed of the airflow, through the device 140. This rotation speed is compared with a look-up table of rotation timer values to determine airflow characteristics, including peak expiratory flow rate (see block 530 of the method 500 of FIG. 5). The look-up table may be stored in the memory 430 and used by the processor 420 to determine the airflow characteristics.

The rotatable body 1015, shaft 1017, and magnet 1010 form an assembly that is mounted in and supported by the frame 1070. This assembly is rotatable relative to the frame 1070 but is constrained from lateral or longitudinal movement relative thereto. The frame 1070 includes a nozzle 1071 through which medication may be dispensed to the patient 130 from a metered dose applicator 1005. The metered dose applicator 1005 stores the medication that is dispensed by the device 140. In some embodiments, the metered dose applicator 1005 is removable and is replaced when a predetermined number of does of the medication stored thereby have been used.

As discussed above, the device 140 may also be used by a patient as a conventional inhaler. The metered dose applicator 1005 includes an aerosol canister body 1006 containing a medication-containing aerosol (or simply “medication”) for the treatment of asthma. The aerosol canister body 1006 includes a valve stem 1007 extending downward therefrom. When the valve stem 1007 is pressed toward the canister body 1006, a metered amount of the medication-containing aerosol is released from the valve stem. As shown in FIGS. 11 and 12, the metered dose applicator 1005 may be positioned within a chamber 1032 formed by an upwardly extending sidewall 1031 of the main enclosure body 1030. In this installed position, the valve stem 1007 is positioned within the nozzle 1071. In some embodiments, the metered dose applicator 1005 and portions of the main enclosure body 1030 may be configured using conventional inhaler components.

During use as an inhaler, a patient may position his/her mouth over a patient mouthpiece portion 1025 of the main enclosure body 1030. The patient may then manually depress the canister body 1006 while inhaling, which causes the valve stem 1007 to be pushed into the canister body. The medication-containing aerosol is released from the valve stem 1007 into the nozzle 1071, where it then travels through a channel 1026 in the main enclosure body 1030 to be ejected out of the patient mouthpiece portion 1025 of the enclosure body so it may be inhaled by the patient. As discussed above with reference to the method 500 of FIG. 5, when the patient is inhaling, the device 140 senses this and the inhalation is assumed to be a medication dispensing event that may be stored and/or transmitted as described above.

During use as an airflow meter, a patient may position his/her mouth over the patient mouthpiece portion 1025 and exhale into the mouthpiece, causing air to flow into the channel 1026 whereby the rotatable body 1015 is rotated at a speed dependent on the rate of airflow. As discussed above with reference to the method 500 of FIG. 5, the device 140 senses when the patient is exhaling, and proceeds to measure airflow characteristics (e.g., peak expiratory flow rate), which may then be stored and/or transmitted as described above.

The frame 1070 also retains a seal plug 1065 in the main enclosure body 1030. This provides a waterproof seal to an electronics enclosure 1067. The seal plug 1065 also allows the device to be washed. A cover 1020 may be positioned over the patient mouthpiece 1025 when the device 140 is not in use. The shape and size of the completed enclosure is intended to mate with standard spacer tubes that are currently on the market and commonly used by children who are less able to time the device 140 spray with their own inhalation.

In an alternate embodiment, the device 140 may include two pressure sensors, instead of the low and high resolution sensors 425 and 435. In such an embodiment, one of the pressure sensors is positioned in a cavity that opens into the airway of the patient 130 and the other pressure sensor communicates with a venturi meter that joins the cavity with the outside air. Such a device can be configured to sense and differentiate inhalation and exhalation airflow and airflow rates.

There are several variations of this device 140 that may be constructed depending on the requirements of the patient 130. For a patient with a modest disease condition, the radio 410 may not be required. In this regard, simply indicating with the display 415 (e.g., via the green, yellow, and red LEDs) the results of the comparison of the current peak exhalation airflow measurement and the comparison measurement may be satisfactory. On the other hand, a patient 130 with a more severe disease condition may prefer the control system 120 features described above. The optional pressure sensor 445 (see FIG. 4) may be used to improve precision in cases of changing altitude or weather, which may alter the measurements by the low and high resolution sensors 425 and 435 due to changes in air pressure.

By integrating an airflow meter with a medication dispensing device, several significant advantages over current art are achieved. Although it is a common recommendation to perform regular peak exhalation flow measurements, it can be difficult in practice. It is inconvenient to carry an additional object. And it is recommended to use the flow meter even when the patient is feeling well. By integrating the medication administration device and the airflow meter into a single device, the patient 130 is more likely to have the flow measurement device and more likely to use it because the patient may already have it in hand. This device 140 places the flow meter in the mouth of the patient 130, who only needs to exhale to obtain an airflow measurement. This also facilitates incentive spirometry activities. Further, the device 140 may be configured to be in a sleep state when not in use. This allows the device 140 to recognize airflow events without requiring the patient to perform any turn on or turn off actions making the device even more convenient to use.

Recommendations for peak exhalation airflow measurement sometimes suggest that the patient 130 take several tests and choose the largest number for logging purposes. The processor 420 may be configured to evaluate a series of exhalation airflow measurements taken in close succession, automatically identify the largest measurement in the series, and transmit the identified measurement (via the radio 410) to the control system 120 for storage in the patient record on the database server 375.

The device 140 and/or the database server 375 may also track the number of dosages used from a metered dose inhaler. In such embodiments, the metered dose applicator 1005 may be discarded when it exceeds its manufacturer's specified dosage capacity. In some embodiments, two or more devices 140 may be attached to different metered dose inhalers that contain different medications. In this way, it is possible for the control system 120 to track more than one medication for a particular patient.

By using a camera of the patient cellular phone with an appropriate bar code reader program, the medication can be scanned, and the drug type, dosage, counter zeroing, and other similar data storage and application tasks may be performed.

The camera of the patient cellular phone 360 may, optionally, also be actuated to gather additional environmental information when a rescue inhaler has been used. This picture may be forwarded to the database server 375 and may be analyzed for commonalities of environmental triggers.

The radio 410 of the device 140 provides a data link to the cellular phone 360 and the control system 120. In some embodiments, to link the radio 410 to the cellular phone 360, it may be necessary to pair the two devices. This is particularly the case with Bluetooth and may be accomplished with this device 140, for example, by blowing through the device 140 while it and the patient's cellular phone 360 are in Bluetooth pairing mode. Similarly, the device 140 may be subjected to several cycles of inhalation and exhalation in quick succession to instruct the processor 420 to clear the retained memory 430 of data regarding peak exhalation flow measurements and dosage specifications. This technique may be referred to as “breath signaling.” In addition to these types of controls, the device 140 may also include one or more inputs (e.g., buttons, keys, etc.) that are operative to allow the user to control the circuit 400 and/or to control the communication of data with the control system 120.

### Computing Device

FIG. 9 is a diagram of hardware and an operating environment in conjunction with which implementations of the database server 375, the patient desktop computer 350, the patient cellular telephone 360, the patient portable computer 365, the support computing device 310, the caregiver computing device 315, and the web server 340 may be practiced. The description of FIG. 9 is intended to provide a brief, general description of suitable computer hardware and a suitable computing environment in which implementations may be practiced. Although not required, implementations are described in the general context of computer-executable instructions, such as program modules, being executed by a computer, such as a personal computer. Generally, program modules include routines, programs, objects, components, data structures, etc., that perform particular tasks or implement particular abstract data types.

Moreover, those skilled in the art will appreciate that implementations may be practiced with other computer system configurations, including hand-held devices, multiprocessor systems, microprocessor-based or programmable consumer electronics, network PCs, minicomputers, mainframe computers, and the like. Implementations may also be practiced in distributed computing environments where tasks are performed by remote processing devices that are linked through a communications network. In a distributed computing environment, program modules may be located in both local and remote memory storage devices.

The exemplary hardware and operating environment of FIG. 9 includes a general-purpose computing device in the form of a computing device 12. The database server 375, the patient desktop computer 350, the patient cellular telephone 360, the patient portable computer 365, the support computing device 310, the caregiver computing device 315, the web server 340 may each be implemented using one or more computing devices like the computing device 12.

The computing device 12 includes a system memory 22, the processing unit 21, and a system bus 23 that operatively couples various system components, including the system memory 22, to the processing unit 21. There may be only one or there may be more than one processing unit 21, such that the processor of computing device 12 includes a single central-processing unit (“CPU”), or a plurality of processing units, commonly referred to as a parallel processing environment. When multiple processing units are used, the processing units may be heterogeneous. By way of a non-limiting example, such a heterogeneous processing environment may include a conventional CPU, a conventional graphics processing unit (“GPU”), a floating-point unit (“FPU”), combinations thereof, and the like. The computing device 12 may be a conventional computer, a distributed computer, or any other type of computer.

The system bus 23 may be any of several types of bus structures including a memory bus or memory controller, a peripheral bus, and a local bus using any of a variety of bus architectures. The memory 430 (illustrated FIG. 4) may be substantially similar to the system memory 22. The system memory 22 may also be referred to as simply the memory, and includes read only memory (ROM) 24 and random access memory (RAM) 25. A basic input/output system (BIOS) 26, containing the basic routines that help to transfer information between elements within the computing device 12, such as during start-up, is stored in ROM 24. The computing device 12 further includes a hard 5 disk drive 27 for reading from and writing to a hard disk, not shown, a magnetic disk drive 28 for reading from or writing to a removable magnetic disk 29, and an optical disk drive 30 for reading from or writing to a removable optical disk 31 such as a CD ROM, DVD, or other optical media.

The hard disk drive 27, magnetic disk drive 28, and optical disk drive 30 are connected to the system bus 23 by a hard disk drive interface 32, a magnetic disk drive interface 33, and an optical disk drive interface 34, respectively. The drives and their associated computer-readable media provide nonvolatile storage of computer-readable instructions, data structures, program modules, and other data for the computing device 12. It should be appreciated by those skilled in the art that any type of computer-readable media which can store data that is accessible by a computer, such as magnetic cassettes, flash memory cards, solid state memory devices (“SSD”), USB drives, digital video disks, Bernoulli cartridges, random access memories (RAMs), read only memories (ROMs), and the like, may be used in the exemplary operating environment. As is apparent to those of ordinary skill in the art, the hard disk drive 27 and other forms of computer-readable media (e.g., the removable magnetic disk 29, the removable optical disk 31, flash memory cards, SSD, USB drives, and the like) accessible by the processing unit 21 may be considered components of the system memory 22.

A number of program modules may be stored on the hard disk drive 27, magnetic disk 29, optical disk 31, ROM 24, or RAM 25, including an operating system 35, one or more application programs 36, other program modules 37, and program data 38. A user may enter commands and information into the computing device 12 through input devices such as a keyboard 40 and pointing device 42. Other input devices (not shown) may include a microphone, joystick, game pad, satellite dish, scanner, touch sensitive devices (e.g., a stylus or touch pad), video camera, depth camera, or the like. These and other input devices are often connected to the processing unit 21 through a serial port interface 46 that is coupled to the system bus 23, but may be connected by other interfaces, such as a parallel port, game port, a universal serial 5 bus (USB), or a wireless interface (e.g., a Bluetooth interface). A monitor 47 or other type of display device is also connected to the system bus 23 via an interface, such as a video adapter 48. In addition to the monitor, computers typically include other peripheral output devices (not shown), such as speakers, printers, and haptic devices that provide tactile and/or other types physical feedback (e.g., a force feed back game controller).

The input devices described above are operable to receive user input and selections. Together the input and display devices may be described as providing a user interface. The input devices may be used to receive information from the patient 130, the support person 305, the caregiver 320, and the like. The user interface may be used to display messages (e.g., notifications and alerts) to the patient 130, the support person 305, the caregiver 320, and the like.

The computing device 12 may operate in a networked environment using logical connections to one or more remote computers, such as remote computer 49. These logical connections are achieved by a communication device coupled to or a part of the computing device 12 (as the local computer) Implementations are not limited to a particular type of communications device.

The remote computer 49 may be another computer, a server, a router, a network PC, a client, a memory storage device, a peer device or other common network node, and typically includes many or all of the elements described above relative to the computing device 12. The remote computer 49 may be connected to a memory storage device 50. The logical connections depicted in FIG. 10 include a local-area network (LAN) 51 and a wide-area network (WAN) 52. Such networking environments are commonplace in offices, enterprise-wide computer networks, intranets and the Internet.

Those of ordinary skill in the art will appreciate that a LAN may be connected to a WAN via a modem using a carrier signal over a telephone network, cable network, cellular network, or power lines. Such a modem may be connected to the computing device 12 by a network interface (e.g., a serial or other type of port). Further, many laptop computers may connect to a network via a cellular data modem.

When used in a LAN-networking environment, the computing device 12 is connected to the local area network 51 through a network interface or adapter 53, which is one type of communications device. When used in a WAN networking environment, the computing device 12 typically includes a modem 54, a type of communications device, or any other type of communications device for establishing communications over the wide area network 52, such as the Internet.

The modem 54, which may be internal or external, is connected to the system bus 23 via the serial port interface 46. In a networked environment, program modules depicted relative to the personal computing device 12, or portions thereof, may be stored in the remote computer 49 and/or the remote memory storage device 50. It is appreciated that the network connections shown are exemplary and other means of and communications devices for establishing a communications link between the computers may be used.

The computing device 12 and related components have been presented herein by way of particular example and also by abstraction in order to facilitate a high-level view of the concepts disclosed. The actual technical design and implementation may vary based on particular implementation while maintaining the overall nature of the concepts disclosed.

The memory of the database server 375 stores computer executable instructions that when executed by one or more processors cause the one or more processors to perform all or portions of the methods 700, 750, and/or 800.

The memory 430 of the device 10 stores processor executable instructions that when executed by the processor 420 cause the processor to perform all or portions of the methods 500, 600, 650, 750, and/or 800.

Any of the instructions described above, including the instructions stored by the memory of the database server 375 and in the memory 430 of the device 140, may be stored on one or more non-transitory computer-readable media.

The patient 130 may choose to ignore, interpret, or follow the messages or instructions received from the control system 120. But, this networking of asthma medication administration and patient airflow testing provide numerous advantages over the current art. Adherence to a medication treatment plan is likely to improve if it is measured and adapted. Often the simple act of monitoring human behavior can change it. Also, particularly in the case of a child whose understanding and resolve can be limited, providing real-time data to the support network 310 that includes a parent may improve compliance. Compliance may be monitored by the recognition of insufficient usage of a maintenance medication or the overuse of a rescue inhaler. The system can function as a medication reminder for those patients with a consistent dosage treatment plan who miss an application or overuse a medicine.

It is often difficult for a patient to remember the exact amount of medicine taken and when it was taken when answering a doctor's questions. By automatically providing trended data to the healthcare provider, he or she can review the effectiveness of the treatment plan and make more careful adjustments. In many cases, the patient data review and adjustment of treatment plan may be accomplished without a visit to a practitioner, which may save medical cost and improve healthcare. More generally, the control system 120 shown in FIGS. 1 and 2 and embodied in a device network shown in FIG. 3 allows the doctor improved monitoring and superior control of their patient's disease.

In addition to medication administration and airflow analysis, in the case of an asthma attack, additional information can be collected. As discussed above, some environmental information is available on the Internet. Weather information such as temperature, humidity, wind born small particles, ozone levels, air pollution, pollen count and the like are available. This information can be localized by use of the patient's cellular phone 360, by either cell tower signal location 330 or global positioning system. This additional data may be used to modify a patient's treatment plan and/or to identify causes of changes in a patient's asthma symptoms. A bronchodilator or rescue inhaler administration could cause the control system 120 to capture this localized environmental data available on the web and store it with the patient's electronic record on the server 375. Additionally, the patient 130 may receive a request for additional information from the server 375 to log additional characteristics of the event or events. In some embodiments, a bronchodilator or rescue inhaler administration and/or a severely low peak expiratory flow measurement may cause the device 140 to send a message to the patient cellular phone 360 or other device to automatically display the treatment plan protocol for attack events.

With this detailed history of the exact patient record, it is possible to determine an approximate level of risk of an asthma attack. This patient record or history data may include medication administration, airway flow, attack history, attack location history, environmental conditions, patient responses and the like. The patient 130 may optionally be tracked and his situation evaluated continuously for conditions that might have historically contributed to an attack. This level of risk could be communicated to the patient's cellular phone 360 or other devices (e.g., a support network computing device or a healthcare provider computing device) using messages on a continually updated basis. As a non-limiting example, a person with a particular susceptibility to pollen and windborne particulates may have that data automatically evaluated and downloaded to their cellular phone 360 depending on their location and weather conditions. For example, a changeable icon on a patient's home screen of their cellular phone might change to represent their personal real-time risk factors.

In the case where an asthma attack occurs, the control system 120 may also be able to help. Often a protocol of medicine administration and periods for reapplication or test is specified. A rescue inhaler administration may optionally initiate a program on the cellular phone 360 that reminds the patient 130 of the prescribed treatment plan, sets timers, and questions the patient 130 for details of their condition.

That data then may be immediately transferred to the server database 375 for evaluation. The control system 120 may then take preprogrammed actions that may include contacting the support network 310 or the healthcare system 315 with current health conditions and the location of the cellular phone 360. In this way, as an example, a parent may be automatically notified of a child's asthma attack in close to real-time with GPS location by preprogrammed control system 120 configuration.

The control system 120, as mentioned earlier, can know the amount of medication consumed by the patient 130 and compare it to the treatment plan. But the control system 120 can also use this information to facilitate reordering of medication. It is possible for the patient 130 or their support network 310 to define the amount of safety stock required and to reorder medication automatically or via prompts when it is required through a control system connection to an Internet-enabled pharmacy.

In addition to the supervisory function of the control system 120, it can also function as a means of providing information and education to the patient. In an optional implementation, the database server 375 may send messages to the patient's cell phone 360 or the support network person 305 to educate and generally inform them about asthma. In addition to messages, links may also be made available to a patient's detailed drug information, counter indications, and the like. As can be appreciated, there is much that the patient 130 can do to improve their condition through a better understanding of how to cope with their disease.

The foregoing described embodiments depict different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present.

For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Accordingly, the invention is not limited except as by the appended claims.

The invention claimed is:

1. An airflow measurement device for use by a patient and for use with an external device, the airflow measurement device comprising:
a metered dose medication applicator;
a processor;
a sensor coupled to the processor and operative to measure air movement through the metered dose medication applicator caused by the patient; and
a communication mechanism coupled to the processor and operative to transmit the air movement measurements to the external device.

2. The airflow measurement device of claim 1, wherein the sensor comprises a magnetic mechanical component operative to encode air movement and a magnetic detector operative to detect movement of the magnetic mechanical component.

3. The airflow measurement device of claim 1, where a rotatable magnetic body encodes the direction of air movement by magnetic communication with the sensor.

4. The airflow measurement device of claim 1, further comprising a memory coupled to the processor and operative to store at least one airflow measurement reading.

5. The airflow measurement device of claim 4, further comprising a display, wherein the processor is configured to compare a current airflow measurement reading with the at least one airflow measurement reading, and to display information pertaining to the result of the comparison on the display.

6. The airflow measurement device of claim 5, wherein the display comprises a plurality of differently-colored light emitting diodes (LEDs), and the information comprises color-coded messages displayed by the plurality of differently-colored LEDs.

7. The device of claim 4, wherein the processor is configured to receive signals from the sensor, and in response, to after or dear the memory based on the signals.

8. The airflow measurement device of claim 1, wherein the air movement measurements are automatically transmitted to the external device without user intervention.

9. The airflow measurement device of claim 1, wherein the processor is configured to receive a plurality of airflow measurements closely associated in time and to determine a peak expiratory airflow rate dependent on the received plurality of airflow measurements.

10. The airflow measurement device of claim 1, wherein the processor is configured to track medication data including specified dosage capacity of a container of medication, an expiration date of the container of medication, and usage data for the container of medication, the communication mechanism being further operative to wirelessly transmit the medication data to the external device.

11. The airflow measurement device of claim 1, wherein the communications mechanism is operative to use breath signaling by the patient to pair the airflow measurement device with one or more wireless external devices.

12. A system for use with an external device and an asthma monitoring device of a patient, at least one of the external device and the asthma monitoring device comprising a display device, the external device and the asthma monitoring device each being connected to the system by at least one network, the asthma monitoring device being operable to collect at least one airflow measurement of the patient and transmit the at least one airflow measurement to the system via the at least one network, the asthma monitoring device being further operable to collect medication application data and transmit the medication application data to the system via the at least one network, the system comprising:
one or more processors;
one or more storage devices connected to the one or more processors, each of the one or more storage devices storing at least one of data and instructions, the data comprising a predetermined treatment plan for treating an asthma condition of the patient, the instructions being executable by the one or more processors and when executed thereby implement a method comprising:
receiving state information from the asthma monitoring device comprising at least one of medication application data and airflow measurement data;
comparing the state information with reference information comprising at least one of the predetermined treatment plan and historical data for the patient;
determining whether the state information deviates from the reference information based on the comparison;
if the one or more processors determine the state information has deviated from the reference information in one or more predetermined ways, the method further comprising:
sending a first message over the at least one network to at least one of the external device and the asthma monitoring device for display thereby on the display device, the first message comprising information directed to the patient concerning the patient's asthma condition.

13. The system of claim 12, wherein the method implemented by the processor executing the instructions further comprises delivering information relating to the patient's asthma condition to at least one of a support network computing device and a healthcare provider computing device.

14. The system of claim 12, wherein the method implemented by the processor executing the instructions further comprises providing airflow measurement data to at least one of a support network computing device and a healthcare provider computing device.

15. The system of claim 12, wherein the reference information comprises the predetermined treatment plan and the state information comprises current and historical patient data, and wherein the method implemented by the processor executing the instructions further comprises sending messages to external devices of the patient and the patient's support network or healthcare provider pertaining to the patient's adherence to the predetermined treatment plan.

16. The system of claim 12, wherein the system is operative for use with a rescue inhaler of the patient that is connected to the system by the at least one network, the rescue inhaler being operable to collect usage data and to transmit the usage data to the system via the at least one network, wherein the usage of the rescue inhaler triggers the system to log data received from the external device and the Internet pertaining to at least one of: current location of the external device, time of day, date, temperature, humidity, wind speed, rain, pollen count, ozone level, air pollution, and an image taken by the external device.

17. The system of claim 16, wherein the method implemented by the processor executing the instructions further comprises comparing the data logged by the system with current conditions at the location of the patient and sending messages to external devices of the patient and the patient's support network or healthcare provider based on the results of the comparison.

18. The system of claim 17, wherein the results of the comparison are made available as a changeable icon on the display of one or more of the external devices.

19. The system of claim 12, wherein the method implemented by the processor executing the instructions further comprises sending messages to a support network computing device when it is determined that the patient's airflow measurements are below a predetermined level.

20. The system of claim 12, wherein the method implemented by the processor executing the instructions further comprises:
receiving usage information for a particular medication from the asthma monitoring device;
comparing the received usage information to a specified usage capacity for the medication;
determining when the medication is at or near its specified usage capacity based on the comparison; and
initiating an order for a replacement medication that is to be provided to the patient.

21. An airflow measurement device for use with one or more external devices, the airflow measurement device comprising:
a metered dose medication applicator; and
a circuit connecting a processor to one or more sensors and a communication mechanism, the one or more sensors being operative to measure airflow characteristics including direction and flow rate, the processor being configured to:
instruct the one or more sensors to measure airflow characteristics;
receive airflow measurements from the one or more sensors; and
instruct the communication mechanism to wirelessly transmit to the one or more external devices the airflow measurements received from the one or more sensors.

22. The airflow measurement device of claim 21, further comprising:
a display device; and
a memory configured to store a plurality of airflow measurements received from the one or more sensors, wherein the processor is further configured to compare a current airflow measurement to a previously stored airflow measurement, and to instruct the display device to display information to a user of the airflow measurement device based on the comparison.

23. The airflow measurement device of claim 22, wherein the display device comprises a plurality of differently-colored light emitting diodes (LEDs).

24. The airflow measurement device of claim 21, wherein the one or more sensors comprise Hall Effect sensors.

25. The airflow measurement device of claim 21, wherein the one or more sensors comprise a low resolution sensor operable to detect airflow, and in response to detecting airflow, to send a signal to the processor, the processor being operable to transition from a sleep state to a working state in response to receiving the signal from the low resolution sensor.

26. The airflow measurement device of claim 21, wherein the communication mechanism comprises a radio connected to an antenna, the radio being configured to transmit the airflow measurement information to the one or more external devices wirelessly via the antenna.

27. An asthma monitoring device, comprising:
a metered dose medication applicator;
a display device;
a sensor configured to detect an airflow measurement indicating an expiratory flow rate of a patient; and
at least one processor configured to:
determine whether the expiratory flow rate has changed based on a currently detected airflow measurement and at least one previously detected airflow measurement, and
instruct the display device to display information indicating the current expiratory flow rate has changed.

28. The asthma monitoring device of claim 27, further comprising:
a memory configured for storing at least one trigger value corresponding to a change in expiratory flow rate, wherein the at least one processor is further configured to:
determine whether the expiratory flow rate has changed by at least the trigger value, and
instruct the display device to display information to the patient that conveys information pertaining to the patient's current expiratory flow rate relative to a previously determined expiratory flow rate.

29. The asthma monitoring device of claim 27, wherein the processor is operative to selectively transition between a low power state and a normal operating state in response to receiving a signal from the sensor indicating a presence or absence of airflow, such that the asthma monitoring device does not require a separate means for turning it on and turning it off.

30. A system for use with an external device and an asthma monitoring device of a patient, at least one of the external device and the asthma monitoring device comprising a display device, the external device and the asthma monitoring device each being connected to the system by at least one network, the asthma monitoring device being operable to collect at least one airflow measurement of the patient and transmit the at least one airflow measurement to the system via the at least one network, the asthma monitoring device being further operable to collect medication application data and transmit the medication application data to the system via the at least one network, the system comprising:
one or more processors;
one or more storage devices connected to the one or more processors, each of the one or more storage devices storing at least one of data and instructions, the data comprising data relating to an asthma condition of the patient, the instructions being executable by the one or more processors and when executed thereby implement a method for recognizing changes in the patient's asthma condition and providing real-time feedback to the patient or an entity associated with the patient to reduce the severity of the patient's current asthma condition, the method comprising:
receiving airflow measurement data from the asthma monitoring device over the at least one network;
comparing the airflow measurement data with one or more preconfigured trigger values; and
if the one or more processors determine the airflow measurement data has exceeded one or more of the trigger values in one or more predetermined ways, the method further comprising:
sending a first message over the at least one network to at least one of the external device and the asthma monitoring device for display thereby on the display device, the first message comprising information directed to the patient or to the entity associated with the patient concerning the patient's asthma condition.

31. An airflow measurement device for use by a patient and for use with an external device, the airflow measurement device comprising:
a metered dose medication applicator;
a processor;
a sensor coupled to the processor and operative to measure air movement through the metered dose medication applicator caused by the patient;
a communication mechanism coupled to the processor and operative to transmit the air movement measurements to the external device, the external device being operative to transmit the air movement measurements to a remote device over at least one network, and to receive and display patient instructions received from the remote device over the at least one network.
